(19) United States
(12) Patent Application Publication
(10) Pub. No.: US 2003/0143606 A1
(43) Pub. Date: Jul. 31, 2003

(54) Diagnosis of diseases associated with the immune system by determining cytosine methylation

(76) Inventors: Alexander Olek, Berlin (DE); Kurt Berlin, Stahnsdorf (DE); Christian Piepenbrock, Berlin (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/311,455

(22) PCT Filed: Jul. 2, 2001
(86) PCT No.: PCT/EP01/07537

(30) Foreign Application Priority Data
Jun. 30, 2000 (DE) 10032529.7
Sep. 1, 2000 (DE) 10043826.1

(51) Int. Cl. C12Q 1/68; C07H 21/04; C12P 19/34

(52) U.S. Cl. 435/6; 435/91.2; 536/24.3

(57) ABSTRACT

The present invention relates to chemically modified genomic sequences of genes associated with the immune system, to oligonucleotides and/or PNA-oligomers directed against the sequence, for the detection of the methylation status of genes, associated with the immune system as well as to a method for ascertaining genetic and/or epigentic parametres of genes, associated with the immune system.

### FIELD OF THE INVENTION

The levels of observation in molecular biology which have been studied well after the methodical developments of the recent years, are the genes themselves, the translation of these genes into RNA, and the proteins resulting therefrom. The question of which gene is switched on at which point in the course of the development of an individual, and the question of how the activation and inhibition of specific genes in specific cells and tissues are controlled is correlatable to the degree and character of the methylation of the genes or of the genome. In this respect, pathogenic conditions manifest themselves in a changed methylation pattern of individual genes or of the genome.

The present invention relates to nucleic acids, oligonucleotides, PNA-oligomers and to a method for the diagnosis and/or therapy of diseases which have a connection with the genetic and/or epigenetic parameters of genes associated with the immune system and, in particular, with the methylation status thereof.

### PRIOR ART

Very many human diseases are associated with the immune system. The immune system recognises micororganisms (bacteria, viruses, fungi) invading the body and disarms these. A distinction is drawn between two systems working closely together. The so called non-specific, humoral blocking system generally directs against pathogens invaded and—independently concerning the kind of pathogen and the causing disease—tries to kill them. The second system is the specific cellular blocking system. It acts much more specifically against pathogens by producing antibodies according to the structure of the particular pathogen helping to overcome the disease. Particular pathogens are recognised when appearing once again and are more rapidly eliminated; in many cases the organism is immune for a lifetime. However, diseases associated with the immune system are not only related to disease patterns developed by pathogens and generally being fought successfully by a healthy immune system. With many chronic diseases like rheumatism or asthma a so called immunodeficiency is basically involved in the cause of the disease. Last but not least stress and other mental impacts have a negative influence on the immune system.

Diseases, caused by false or overreaction of an intact immune system are integrated in the generic terms allergies, like e.g. asthma (Kuo M L, Huang J L, Yeh K W, Li P S, Hsieh K H. Evaluation of Th1/Th2 ratio and cytokine production profile during acute exacerbation and convalescence in asthmatic children. Ann Allergy Asthma Immunol. 2001 Mar;86(3):272-6) and autoimmune diseases like e.g. arteriosclerosis (Gordon P A, George J, Khamashta M A, Harats D, Hughes G, Shoenfeld Y. Arteriosclerosis and autoimmunity. Lupus. 2001;10(4):249-52), systemic lupus erythematosus (Lorenz H M, Herrmann M, Winkler T, Gaipl U, Kalden J R. Role of apoptosis in autoimmunity. Apoptosis. 2000 Nov;5(5):443-9) or Type I Diabetes mellitus (Not T, Tommasini A, Tonini G, Buratti E, Pocecco M, Tortul C, Valussi M, Crichiutti G, Berti I, Trevisiol C, Azzoni E, Neri E, Torre G, Martelossi S, Soban M, Lenhardt A, Cattin L, Ventura A. Undiagnosed coeliac disease and risk of autoimmune disorders in subjects with Type I diabetes mellitus. Diabetologia. 2001 Feb;44(2):151-5). There are also several correlations between the immune system and cancer diseases like anemia (Bron D, Meuleman N, Mascaux C. Biological basis of anemia. Semin Oncol. 2001 Apr;28(2 Suppl 8):1-6), pancreatic carcinoma (Shimura T, Tsutsumi S, Hosouchi Y, Kojima T, Kon Y, Yonezu M, Kuwano H. Clinical significance of soluble form of HLA class I molecule in Japanese patients with pancreatic cancer. Hum Immunol. 2001 Jun;62(6):615-9), chronic myelogenous leukaemia (Jahagirdar B N, Miller J S, Shet A, Verfaillie C M. Novel therapies for chronic myclogenous leukaemia. Exp Hematol. 2001 May;29(5):543-56), acute lymphoblastic leukaemia (Velders M P, ter Horst S A, Kast W M. Prospect for immunotherapy of acute lymphoblastic leukaemia. Leukaemia. 2001 May;15(5):701-6) or acute myeloid leukaemia (Harrison B D, Adams J A, Briggs M, Brereton M L, Yin J A. Stimulation of autologous proliferative and cytotoxic T-cell responses by “leukaemia dendritic cells” derived from blast cells in acute myeloid leukaemia. Blood. 2001 May 1;97(9):2764-71). The cells of the human immune system recognise many tumour cells for being unfamiliar and try to attack them. With a cancer patient, however, the defence of the body is not able to destroy the tumour. Cancer cells compared to normal body cells show modified characteristics and often also form different proteins that play an important role in the recognition of “self” and “unfamiliar” in the immune system. In the body they are presented to the killer T-cells, bound to MHC molecules at the outside of the cells. Killer T-cells control whether the peptides have their origin from normal proteins. If a peptide is not cut from a normal endogenic protein, the T-cells start destroying the cell, presenting the modified or unfamiliar peptide.

Further diseases associated with the immune system are Alzheimer's disease (Smits H A, van Beelen A J, de Vos N M, Rijsmus A, van der Bruggen T, Verhoef J, van Muiswinkel F L, Nottet H S. Activation of human macrophages by amyloid-beta is attenuated by astrocytes J Immunol. 2001 Jun 1;166(11):6869-76), acquired immune deficiency syndrome (Aids) (McGrath K M, Hoffman N G, Resch W, Nelson J A, Swanstrom R. Using HIV-1 sequence variability to explore virus biology. Virus Res. 2001 Aug;76(2):137-60.), progressive focal epilepsy (Ponomareva E N, Khmara M E, Nedz'ved' M K, Drakina S A, Kolomiets A G, Protas I I [The clinical characteristics of progressive focal epilepsy with a herpetic etiology]. Lik Sprava. 2000 Jul-Aug;(5):106-10), primary sclerosing cholangitis 1 (Bo X, Broome U, Remberger M, Sumitran-Holgersson S. Tumour necrosis factor alpha impairs function of liver derived T lymphocytes and natural killer cells in patients with primary sclerosing cholangitis. Gut. 2001 Jul;49(1):131-41), or neurofibromatosis (Gerosa P L, Spinelli M, Giussani G, Vai C, Fontana A, Canepari C. Neurofibromatosis (NF1) and neuroleprosy: immunoreaction against pathologic Schwann-cells. Physiopathogenetic observations. Minerva Med. 2001 Apr;92(2): 89-97).

Methods of treatment for immune diseases above all concentrate on allergies, autoimmune diseases as well as on the development of vaccines to stimulate stronger immune responses for pathogene organism and cancer (Fahrer A M, Bazan J F, Papathanasiou P, Nelms K A, Goodnow C C. A genomic view of immunology. Nature. 2001 Feb 15;409(6822):836-8).

5-methylcytosine is the most frequent covalently modifiable base in the DNA of eukaryotic cells. It plays a role, for example, in the regulation of the transcription, in genetic imprinting, and in tumorigenesis. Therefore, the identification of 5-methylcytosine as a part of genetic information is of considerable interest. However, 5-methylcytosine positions cannot be identified by sequencing since 5-methylcytosine has the same base pairing behaviour as cytosine. Moreover, the epigenetic information carried by the 5-methylcytosines is completely lost during a PCR amplification.

A relatively new and now the most frequently used method for analysing DNA for 5-methylcytosine is based on the specific reaction of bisulfite with cytosine which, upon subsequent alkaline hydrolysis, is converted to uracil which corresponds to thymidine in its base pairing behaviour. However, 5-methylcytosine is not modified under these conditions. Consequently, the original DNA is converted in such a manner that methylcytosine, which originally cannot be distinguished from cytosine because of its hybridisation behaviour, can now be detected as the only remaining cytosine using “normal” molecular biological techniques, for example, by amplification and hybridisation or sequencing. All these techniques are based on base pairing which is now taken full advantage of. The Prior Art is defined in terms of sensitivity by a method which encloses the DNA to be analysed in an agarose matrix, thus preventing the diffusion and renaturation of the DNA (bisulfite reacts only on single-stranded DNA), and which replaces all precipitation and purification steps with fast dialysis (Olek A, Oswald J, Walter J. A modified and improved method for bisulphite based cytosine methylation analysis. Nucleic Acids Res. 1996 Dec 15;24(24):5064-6). Using this method, it is possible to analyse individual cells, which illustrates the potential of the method. Heretofore, however, only individual regions of a length of up to approximately 3000 base pairs are analysed, a global analysis of cells for thousands of possible methylation analyses is not possible [sic]. However, this method cannot reliably analyse very small fragments from small sample quantities either. These are lost through the matrix in spite of the diffusion protection. An overview of the further known possibilities of detecting 5-methylcytosines can be gathered from the following survey article: Rein, T., DePamphilis, M. L., Zorbas, H., Nucleic Acids Res. 1998, 26, 2255.

Heretofore, the bisulfite technology is only used in research with few exceptions (e.g., Zesch-nigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. 1997 Mar-Apr;5(2):94-8). Always, however, short, specific fragments of a known gene are amplified subsequent to a bisulfite treatment and either completely sequenced (Olek A, Walter J. The pre-implantation ontogeny of the H19 methylation imprint. Nat Genet. 1997 Nov; 17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. 1997 Jun 15;25(12):2529-31, WO Patent 9500669) or by an enzyme cut (Xiong Z, Laird P W. COBRA: a sensitive and quantitative DNA methylation assay. Nucleic Acids Res. 1997 Jun 15;25(12):2532-4). In addition, the detection by hybridisation has also been described (Olek et al., WO 99 28498).

Further publications dealing with the use of the bisulfite technique for methylation detection in individual genes are: Grigg G, Clark S. Sequencing 5-methylcytosine residues in genomic DNA. Bioassays. 1994 Jun;16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. 1997 Mar;6(3):387-95; Feil R, Charlton J, Bird A P, Walter J, Reik W. Methylation analysis on individual chromosomes: improved protocol for bisulphite genomic sequencing. Nucleic Acids Res. 1994 Feb 25;22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. 1995 May 19;157(1-2):261-4; WO 97/46705, WO 95/15373 and WO 97/45560.

An overview of the Prior Art in oligomer array manufacturing can be gathered from a special edition of Nature Genetics (Nature Genetics Supplement, Volume 21, January 1999), published in January 1999, and from the literature cited there.

For scanning an immobilised DNA array, fluorescently labelled probes have often been used. Particularly suitable for fluorescence labels is the simple attachment of Cy3 and Cy5 dyes to the 5′-OH of the specific probe. The detection of the fluorescence of the hybridised probes is carried out, for example via a confocal microscope. Cy3 and Cy5 dyes, besides many others, are commercially available.

Matrix Assisted Laser Desorption Ionization Mass Spectrometry (MALDI-TOF) is a very efficient development for the analysis of biomolecules (Karas M, Hillenkamp F. Laser desorption Ionization of proteins with molecular masses exceeding 10,000 daltons. Anal Chem. 1988 Oct 15;60(20):2299-301). An analyte is embedded in a light-absorbing matrix. By a short laser pulse, the matrix is evaporated, thus transporting the analyte molecule into the vapour phase in an unfragmented manner. The analyte is ionised by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suitable for analysing peptides and proteins. The analysis of nucleic acids is somewhat more difficult (Gut I G, Beck S. DNA and Matrix Assisted Laser Desorption Ionization Mass Spectrometry. Current Innovations and Future Trends. 1995, 1; 147-57). The sensitivity for nucleic acids is approximately 100 times worse than for peptides and decreases superproportionally with increasing fragment size. For nucleic acids having a multiply negatively charged backbone, the Ionization process via the matrix is considerably less efficient. In MALDI-TOF spectrometry, the selection of the matrix plays an eminently important role. For the desorption of peptides, several very efficient matrixes have been found which produce a very fine crystallisation. For DNA, there are indeed several responsive matrixes now, however, the difference in sensitivity has thereby not been reduced. The difference in sensitivity can be reduced by chemically modifying the DNA in such a manner that it becomes more similar to a peptide. Phosphorothioate nucleic acids in which the usual phosphates of the backbone are substituted by thiophosphates can be converted into a charge-neutral DNA using simple alkylation chemistry (Gut I G, Beck S. A procedure for selective DNA alkylation and detection by mass spectrometry. Nucleic Acids Res. 1995 Apr 25;23(8):1367-73). The coupling of a charge tag to this modified DNA results in an increase in sensitivity by the same amount as that found for peptides. A further advantage of charge tagging is the increased stability of the analysis against impurities which make the detection of unmodified substrates considerably more difficult.

Genomic DNA is obtained from DNA of cell, tissue or other test samples using standard methods. This standard methodology is found in references such as Fritsch and Maniatis eds., Molecular Cloning: A Laboratory Manual, 1989.

### PROBLEM DEFINITION

The present invention is intended to provide oligonucleotides and/or PNA-oligomers for detecting cytosine methylations as well as a method which is particularly suitable for the diagnosis and/or therapy of genetic and epigenetic parameters of genes associated with the immune system. The present invention is based on the realisation that, in particular, cytosine methylation patterns are particularly suitable for the diagnosis and/or therapy of diseases associated with the immune system.

### DESCRIPTION

The object of the present invention is to provide the chemically modified DNA of genes associated with the immune system, as well as oligonucleotides and/or PNA-oligomers for detecting cytosine methylations, as well as a method which is particularly suitable for the diagnosis and/or therapy of genetic and epigenetic parameters of genes associated with the immune system. The present invention is based on the realisation that genetic and epigenetic parameters and, in particular, the cytosine methylation pattern of genes associated with the immune system are particularly suitable for the diagnosis and/or therapy of diseases associated with the immune system.

This objective is achieved according to the present invention by a nucleic acid containing an at least 18 bases-long sequence segment of the chemically pretreated DNA of genes associated with the immune system according to one of Seq. ID No.1 through Seq. ID No.2420 and sequences complementary thereto and/or oligonucleotide- and/or PNA-oligomers according to table 1. In the table, after the listed gene designations, the respective data bank numbers (accession numbers) are specified which define the appertaining gene sequences as unique. Gen-Bank was used as the underlying data bank, the internet address thereof is http://www.ncbi.nlm.nih.gov.

The chemically modified nucleic acid could heretofore not be connected with the ascertainment of genetic and epigenetic parameters.

The object of the present invention is further achieved by an oligonucleotide or oligomer for detecting the cytosine methylation state in chemically pretreated DNA, containing at least one base sequence solved having a length of at least 13 nucleotides which hybridises to a chemically pretreated DNA of genes associated with the immune system according to Seq. ID No.1 through Seq. ID No.2420 and sequences complementary thereto and/or oligonucleotide- and/or PNA-oligomers according to table 1. The oligomer probes according to the present invention constitute important and effective tools which, for the first time, make it possible to ascertain the genetic and epigenetic parameters of genes associated the immune system. The base sequence of the oligomers preferably contains at least one CpG dinucleotide. The probes can also exist in the form of a PNA (peptide nucleic acid) which has particularly preferred pairing properties. Particularly preferred are oligonucleotides according to the present invention in which the cytosine of the CpG dinucleotide is the 5th-9th nucleotide from the 5′-end of the 13-mer; in the case of PNA-oligomers, it is preferred for the cytosine of the CpG dinucleotide to be the 4th-6th nucleotide from the 5′-end of the 9-mer.

The oligomers according to the present invention are normally used in so-called sets which contain at least one oligomer for each of the CpG dinucleotides one of the sequences of Seq. ID No.1 through Seq. ID No.2420 and sequences complementary thereto and/or oligonucleotide- and/or PNA-oligomers according to table 1. Preferred is a set which contains at least one oligomer for each of the CpG dinucleotides from one of Seq. ID No.1 through Seq. ID No.2420 and sequences complementary thereto and/or oligonucleotide- and/or PNA-oligomers according to table 1.

Moreover, the present invention makes available a set of at least two oligonucleotides which can be used as so-called primer oligonucleotides for amplifying DNA sequences of one of Seq. ID No.1 through Seq. ID No.2420 and sequences complementary thereto and/or oligonucleotide- and/or PNA-oligomers according to table 1, or segments thereof.

In the case of the sets of oligonucleotides according to the present invention, it is preferred that at least one oligonucleotide is bonded to a solid phase.

The present invention moreover relates to a set of at least 10 n (oligonucleotides and/or PNA-oligomers) used for detecting the cytosine methylation state in chemically pretreated genomic DNA (Seq. ID No.1 through Seq. ID No.2420 and sequences complementary thereto and/or oligonucleotide- and/or PNA-oligomers according to table 1). These probes enable diagnosis and/or therapy of genetic and epigenetic parameters of genes associated with the immune system. The set of oligomers can also be used for detecting single nucleotide polymorphisms (SNPs) in the chemically pretreated DNA of genes associated with the immune system according to one of Seq. ID No.1 through Seq. ID No.2420 and sequences complementary thereto and/or oligonucleotide- and/or PNA-oligomers according to table 1.

According to the present invention, it is preferred that an arrangement of different oligonucleotides and/or PNA-oligomers (a so-called “array”) made available by the present invention is present in a manner that it is likewise bonded to a solid phase. This array of different oligonucleotide- and/or PNA-oligomer sequences can be characterised in that it is arranged on the solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics such as nylon which can exist in the form of pellets or also as resin matrices are possible as well.

Therefore, a further subject matter of the present invention is a method for manufacturing an array fixed to a carrier material for analysis in connection with diseases associated with the immune system in which method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for analysis in connection with diseases associated with the immune system which DNA chip contains at least one nucleic acid according to the present invention. DNA chips are known, for example, from U.S. Pat. No. 5,837,832.

Moreover, a subject matter of the present invention is a kit which can be composed, for example, of a bisulfite-containing reagent, a set of primer oligonucleotides containing at least two oligonucleotides whose sequences in each case correspond or are complementary to an 18 bases-long segment of the base sequences specified in the appendix (Seq. ID No.1 through Seq. ID No.2420 and sequences complementary thereto and/or oligonucleotide- and/or PNA-oligomers according to table 1), oligonucleotides and/or PNA-oligomers as well as instructions for carrying out and evaluating the described method. However, a kit along the lines of the present invention can also contain only part of the aforementioned components.

The present invention also makes available a method for ascertaining genetic and/or epigenetic parameters of genes associated with the immune system by analysing cytosine methylations and single nucleotide polymorphisms, including the following steps:

In a first method steps, a genomic DNA sample is chemically treated in such a manner that cytosine bases which are unmethylated at the 5′-position are converted to uracil, thymine, or another base which is dissimilar to cytosine in terms of hybridisation behaviour. This will be understood by chemical pretreatment hereinafter.

The genomic DNA to be analysed is preferably obtained form usual sources of DNA such as cells or cell components, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, or combinations thereof.

Preferably used for that is the above described treatment of genomic DNA with bisulfite (hydrogen sulfite, disulfite) and subsequent alkaline hydrolysis which results in a conversion of non-methylated cytosine nucleobases to uracil or to another base which is dissimilar to cytosine in terms of base pairing behaviour.

Fragments from this chemically pretreated DNA are amplified, using sets of primer oligonucleotides according to the present invention, and a, preferably heat-stable polymerase. Because of statistical and practicable considerations, preferably more than ten different fragments having a length of 100-2000 base pairs are amplified. The amplification of several DNA segments can be carried out simultaneously in one and the same reaction vessel. Usually, the amplification is carried out by means of the polymerase chain reaction (PCR).

In a preferred embodiment of the method, the set of primer oligonucleotides includes at least two olignonucleotides whose sequences are each reverse complementary or identical to an at least 18 base-pair long segment of the base sequences specified in the appendix (Seq. ID No.1 through Seq. ID No.2420 and sequences complementary thereto and/or oligonucleotide- and/or PNA-oligomers according to table 1). The primer oligonucleotides are preferably characterised in that they do not contain any CpG dinucleotide.

According to the present invention, it is preferred that at least one primer oligonucleotide is bonded to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminium, steel, iron, copper, nickel, silver, or gold, it being possible for other materials such as nitrocellulose or plastics to be used as well.

The fragments obtained by means of the amplification can carry a directly or indirectly detectable label. Preferred are labels in the form of fluorescence labels, radionuclides, or detachable molecule fragments having a typical mass which can be detected in a mass spectrometer, it being preferred that the produced fragments have a single positive or negative net charge for better detectability in the mass spectrometer. The detection can be carried out and visualized by means of matrix assisted laser desorption/Ionization mass spectrometry (MALDI) or using electron spray mass spectrometry (ESI).

The amplificates obtained in the second method step are subsequently hybridised to a set of oligonucleotides and/or PNA probes of or to an array. In this context, the hybridisation takes place in the manner described in the following. The set used during hybridisation is preferably composed of at least 10 oligonucleotide or PNA-oligomer probes. In the process, the amplificates serve as probes which hybridize to oligonucleotides previously bonded to a solid phase. The non-hybridised fragments are subsequently removed. Said oligonucleotides contain at least one base sequence having a length of 13 nucleotides which is reverse complementary or identical to a segment of the base sequences specified in the appendix, the segment containing at least one CpG dinucleotide. The cytosine of the CpG dinucleotide is the 5th to 9th nucleotide seen from the 5′-end of the 13-mer. One oligonucleotide exists for each CpG dinucleotide. Said PNA-oligomers contain at least one base sequence having a length of 9 nucleotides which is reverse complementary or identical to a segment of the base sequences specified in the appendix, the segment containing at least one CpG dinucleotide. The cytosine of the CpG dinucleotide is the 4th to 6th nucleotide seen from the 5′-end of the 9-mer. One oligonucleotide exists for each CpG dinucleotide.

In the fourth method step, the non-hybridised amplificates are removed.

In the last method step, the hybridised amplificates are detected. In this context, it is preferred that labels attached to the amplificates are identifiable at each position of the solid phase at which an oligonucleotide sequence is located.

According to the present invention, it is preferred that the labels of the amplificates are fluorescence labels, radionuclides, or detachable molecule fragments having a typical mass which can be detected in a mass spectrometer. The mass spectrometer is preferred for the detection of the amplificates, fragments of the amplificates or of probes which are complementary to the amplificates, it being possible for the detection to be carried out and visualized by means of matrix assisted laser desorption/Ionization mass spectrometry (MALDI) or using electron spray mass spectrometry (ESI).

The produced fragments can have a single positive or negative net charge for better detectability in the mass spectrometer. The aforementioned method is preferably used for ascertaining genetic and/or epigenetic parameters of genes associated with the immune system.

The oligomers according to the present invention or arrays thereof as well as a kit according to the present invention are intended to be used for the diagnosis and/or therapy of diseases associated with the immune system by analysing methylation patters of genes associated with the immune system. According to the present invention, the method is preferably used for the diagnosis and/or therapy of important genetic and/or epigenetic parameters within genes associated with the immune system.

The method according to the present invention is used, for example, for the diagnosis and/or therapy of eye diseases, proliferative retinopathy, neovascular glaucoma, solid tumors, tissue inflammations, rheumatic arthritis, diabetic retinopathy, macular degeneration due to neovascularization, psoriasis, arteriosclerosis, inflammatory bowel diseases, ulcerative enteritis, Crohn's disease, and cancers.

The nucleic acids according to the present invention of Seq. ID No.1 through Seq. ID No.2420 and sequences complementary thereto and/or oligonucleotide- and/or PNA-oligomers according to table 1 also can be used for the diagnosis and/or therapy of genetic and/or epigenetic parameters of genes associated with the immune system.

The present invention moreover relates to a method for manufacturing a diagnostic agent and/or therapeutic agent for the diagnosis and/or therapy of diseases associated with the immune system by analysing methylation patterns of genes associated with the immune system, the diagnostic agent and/or therapeutic agent being characterised in that at least one nucleic acid according to the present invention is used for manufacturing it, possibly together with suitable additives and auxiliary agents.

A further subject matter of the present invention relates to a diagnostic agent and/or therapeutic agent for diseases associated with the immune system by analysing methylation patterns of genes associated with the immune system, the diagnostic agent and/or therapeutic agent containing at least one nucleic acid according to the present invention, possibly together with suitable additives and auxiliary agents.

The present invention moreover relates to the diagnosis and/or prognosis of events which are disadvantageous to patients or individuals, in which the important genetic and/or epigenetic parameters within genes associated with the immune system, obtained by means of the present invention, can be compared to another set of genetic and/or epigenetic parameters, the differences obtained in this manner serving as the basis for a diagnosis and/or prognosis of events which are disadvantageous to patients or individuals.

To be understood by the term “hybridisation” along the lines of the present invention is a bond of an oligonucleotide to a completely complementary sequence along the lines of the Watson-Crick base pairings in the sample DNA, forming a duplex structure. To be understood by “stringent hybridisation conditions” are those conditions in which a hybridisation is carried out at 60° C. in 2.5×SSC buffer, followed by several washing steps at 37° C. in a low buffer concentration, and remains stable.

The term “functional variants” denotes all DNA sequences which are complementary to a DNA sequence, hybridizing with the reference sequence under stringent conditions and having an activity similar to the corresponding polypeptide according to the present invention.

Along the lines of the present invention, “genetic parameters” are mutations and polymorphisms of genes associated with the immune system and sequences further required for its regulation. To be designated as mutations are, in particular, insertions, deletions, point mutations, inversions and polymorphisms and, particularly preferred, SNPs (single nucleotide polymorphisms). However, polymorphisms can also be insertions, deletions or inversions.

Along the lines of the present invention, “epigenetic parameters” are, in particular, cytosine methylations and further chemical modifications of DNA bases of genes associated with the immune system and sequences further required for their regulation. Further epigenetic parameters include, for example, the acetylation of histones which, however, cannot be directly analysed using the described method but which, in turn, correlates with the DNA methylation.

In the following, the present invention will be explained in greater detail on the basis of the sequences and examples without being limited thereto.

Seq ID No. 1 through Seq ID No. 2420

Sequences having odd sequence numbers (e.g., Seq. ID No. 1, 3, 5, . . . ) exhibit in each case different sequences of the chemically pretreated genomic DNAs of genes associated with the immune system. Sequences having even sequence numbers (e.g., Seq. ID No. 2, 4, 6, . . . ) exhibit in each case the sequences of the chemically pretreated genomic DNAs of genes associated with the immune system, which sequences beeing complementary to the different sequences (e.g., the complementary sequence to Seq. ID No.1 is Seq. ID No.2, the complementary sequence to Seq. ID No.3 is Seq. ID No.4, etc.).

Seq ID No. 2421 through Seq ID No. 2424

Seq ID No. 2421 through Seq ID No. 2424 show sequences of oligonucleotides, used in the examples.

### EXAMPLE 1

Carrying out the methylation analysis in the gene ESR1 (estrogen receptor) associated with the immune system.

The following example relates to a fragment of the gene ESR1, in which a specific CG-position is to be analysed for methylation.

In the first step, a genomic sequence is treated using bisulfite (hydrogen sulfite, disulfite) in such a manner that all cytosines which are not methylated at the 5-position of the base are changed in such a manner that a different base results with regard to the base pairing behaviour while the cytosines methylated in the 5-position remain unchanged. If bisulfite in the concentration range is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. This converted DNA serves for detecting methylated cytosines. In the second method step, the treated DNA sample is diluted with water or an aqueous solution. Preferably, a desulfonation of the DNA is subsequently carried out. In the third step of the method, the DNA sample is amplified in a polymerase chain reaction, preferably using a heat-resistant DNA polymerase. In the present case, cytosines of the gene ESR1 are analysed. To this end, a defined fragment having a length of 662 bp is amplified with the specific primer oligonucleotides AGGGGGAATTAAATAGAAAGAG (SEQ ID NO: 2421) and CAATAAAACCATCCCAAATACT (SEQ ID NO: 2422). This amplificate serves as a sample which hybridises to an oligonucleotide previously bonded to a solid phase, forming a duplex structure, for example TTTAATTTCGGGTTGTGT (SEQ ID NO: 2423), for the detection of a methylated state and TTTAATTTTGGGTTGTGT (SEQ ID NO: 2424) for the detection of a non-methylated state, wherein the cytosine to be detected being located at position 527 of the amplificate. The detection of the hybridisation product is based on Cy3 and Cy5 flourescently labelled primer oligonucleotides which have been used for the amplification. A hybridisation reaction of the amplified DNA with the oligonucleotide takes place only if a methylated cytosine was present at this location in the bisulfite-treated DNA. Thus, the methylation status of the specific cytosine to be analysed decides on the hybridisation product. In the present case (FIG. 1) for the oligomers in illustration A a non-methylated status and for the oligomers in illustration B a partly methylated status is detected.

### EXAMPLE 2

Diagnosis of diseases associated with the immune system

To relate the methylation patterns to one of the diseases associated with the immune system, it is initially required to analyse the DNA methylation patterns of a group of diseased and of a group of healthy patients. These analyses are carried out, for example, analogously to example 1. The results obtained in this manner are stored in a database and the CpG dinucleotides which are methylated differently between the two groups are identified, for example by labelled probes. It is also possible for the entire methylation status to be analysed simultaneously, and for the patterns to be compared, for example, by clustering analyses which can be carried out, for example, by a computer.

Subsequently, it is possible to allocate the examined patients to a specific therapy group and to treat these patients selectively with an individualized therapy.

Example 2 can be carried out, for example, for the following diseases: asthma, arteriosclerosis, anemia, pancreatic carcinoma, acute myeloid leukaemia, Alzheimer's disease, aids, epilepsy, neurofibromatosis.

TABLE 1
List of the preferred genes, associated with the immune system,
according to the present invention

| | Genbank Accession Nr.. |
|---|---|
| Gen | (http://www.ncbi.nlm.nih.gov) |
| A1BG | T80683 |
| C4A | K02403 |
| C4BPAL2 | X81360 |
| CD1A | M27735 |
| CD20 | L23418 |
| CDR2 | M63256 |
| CENPB | X05299 |
| COL11A2 | U32169 |
| CR1L | M31230 |
| CYP2A | X06401 |
| EBF | AA504812 |
| ERCC2 | L47234 |
| ESD | M13450 |
| ETV4 | D12765 |
| FCGR2C | M90737 |
| FLG | M24355 |
| FN1 | M10905 |
| ITGA1 | X68742 |
| ITGAD | U40274 |
| KRT4 | X07695 |
| KSR | U43586 |
| LY9 | L42621 |
| MEKK1 | U29671 |
| MFAP4 | L38486 |
| MMP18 | Y08622 |
| MYCL1 | M19720 |
| NOTCH1 | M73980 |
| PDE7A | L12052 |
| PIK3R1 | M61906 |
| SLC9A1 | M81768 |
| TCF3 | M31222 |
| TCRA | M12959 |
| TCRB | K02779 |
| TCRG | M27331 |
| TLR5 | U08888 |
| TNFSF11 | AF013171 |
| UBC | AB009010 |
| ZNF121 | M99593 |
| ZRK | L08961 |
| ALPPL2 | NM_031313 |
| AHSG | NM_001622 |
| FCGR3A | NM_000569 |
| FUT3 | NM_000149 |
| IL1R2 | NM_004633 |
| IL2RB | NM_000878 |
| LHB | NM_000894 |
| MDH2 | NM_005918 |
| SLC11A2 | NM_000617 |
| OMG | NM_002544 |
| PIK3CA | NM_006218 |
| TPM1 | NM_000366 |
| TUB | NM_003320 |
| ABAT | NM_000663 |
| ACADL | NM_001608 |
| ACO1 | NM_002197 |
| ADAM10 | NM_001110 |
| ADD1 | NM_014189 |
| ADH4 | NM_000670 |
| ADRA2C | NM_000683 |
| AGA | NM_000027 |
| AGTR2 | NM_000686 |
| AKT1 | NM_005163 |
| ALDH6 | NM_000693 |
| AMPH | NM_001635 |
| ANXA4 | NM_001153 |
| APBA2 | NM_005503 |
| APC | NM_000038 |
| APOA2 | NM_001643 |
| ARHGAP1 | NM_004308 |
| ATOX1 | NM_004045 |
| ATP2B2 | NM_001683 |
| ATP4B | NM_000705 |
| ATR | NM_001184 |
| AUH | NM_001698 |
| AXL | NM_001699 |
| BCL2 | NM_000633 |
| BENE | NM_005434 |
| BID | NM_001196 |
| BMI1 | NM_005180 |
| BN51T | NM_001722 |
| BUB1 | NM_004336 |
| C1R | NM_001733 |
| C4BPB | NM_000716 |
| C5R1 | NM_001736 |
| CASP3 | NM_004346 |
| CASP7 | NM_001227 |
| CBFB | NM_001755 |
| CCR4 | NM_005508 |
| CD151 | NM_004357 |
| CD36L1 | NM_005505 |
| CD4 | NM_000616 |
| CD81 | NM_004356 |
| CDH12 | NM_004061 |
| CDW52 | NM_001803 |
| CEL | NM_001807 |
| CES1 | NM_001266 |
| CGA | NM_000735 |
| CHS1 | NM_000081 |
| CLDN3 | NM_001306 |
| CNK | NM_004073 |
| CSF2RA | NM_006140 |
| CTSK | NM_000396 |
| CX3CR1 | NM_001337 |
| CYBB | NM_000397 |
| CYP11A | NM_000781 |
| DCC | NM_005215 |
| DFFB | NM_004402 |
| DOCK1 | NM_001380 |
| DPYD | NM_000110 |
| ELAVL2 | NM_004432 |
| ELAVL4 | NM_021952 |
| EPB41 | NM_004437 |
| EPHA3 | NM_005233 |
| EPHX2 | NM_001979 |
| EPS15 | NM_001981 |
| ETV6 | NM_001987 |
| F2 | NM_000506 |
| F8A | NM_012151 |
| FABP6 | NM_001445 |
| FADD | NM_003824 |
| FANCE | NM_021922 |
| FCAR | NM_002000 |
| FGA | NM_021871 |
| FGB | NM_005141 |
| FGFR3 | NM_000142 |
| FGG | NM_000509 |
| HFL3 | NM_005666 |
| FOXO1A | NM_002015 |
| ADAM2 | NM_001464 |
| FUCA1 | NM_000147 |
| FUT2 | NM_000511 |
| FY | NM_002036 |
| GABRA5 | NM_000810 |
| GABRA6 | NM_000811 |
| GAS | NM_000805 |
| GAS6 | NM_000820 |
| GBA | NM_000157 |
| GFI1 | NM_005263 |
| GH2 | NM_002059 |
| GHR | NM_000163 |
| GIF | NM_005142 |
| GNAQ | NM_002072 |
| GP9 | NM_000174 |
| GPR15 | NM_005290 |
| GPR30 | NM_001505 |
| GRB14 | NM_004490 |
| GRIK1 | NM_000830 |
| GUCY2D | NM_000180 |
| HADHA | NM_000182 |
| NRG1 | NM_013964 |
| HIVEP1 | NM_002114 |
| HLALS | NM_001531 |
| HLCS | NM_000411 |
| HMX1 | NM_018942 |
| HNRPD | NM_002138 |
| HSA277165 | NM_018411 |
| HRG | NM_000412 |
| HSPG2 | NM_005529 |
| HTN3 | NM_000200 |
| HTR2A | NM_000621 |
| HTR7 | NM_000872 |
| IFNA1 | NM_024013 |
| IL10RA | NM_001558 |
| IL1A | NM_000575 |
| IL1B | NM_000576 |
| IL1R1 | NM_000877 |
| IL3RA | NM_002183 |
| IL9 | NM_000590 |
| ILF1 | NM_004514 |
| ILF2 | NM_004515 |
| SCYB10 | NM_001565 |
| INPP5D | NM_005541 |
| ITGAX | NM_000887 |
| ITGB1 | NM_002211 |
| ITGB3 | NM_000212 |
| ITGB5 | NM_002213 |
| ITGB7 | NM_000889 |
| ITK | NM_005546 |
| KCNJ3 | NM_002239 |
| KPNA1 | NM_002264 |
| LECT2 | NM_002302 |
| LEPR | NM_002303 |
| LPA | NM_005577 |
| KCNH2 | NM_000238 |
| LSP1 | NM_002339 |
| LTF | NM_002343 |
| MAB21L1 | NM_005584 |
| MAL | NM_002371 |
| MASP1 | NM_001879 |
| MCF2 | NM_005369 |
| MAP3K3 | NM_002401 |
| MMP16 | NM_022564 |
| MMP17 | NM_016155 |
| MMP23A | NM_004659 |
| MMP7 | NM_002423 |
| MYC | NM_002467 |
| NAGA | NM_000262 |
| NAT2 | NM_000015 |
| NDUFS2 | NM_004550 |
| NEB | NM_004543 |
| NEU1 | NM_000434 |
| NFATC4 | NM_004554 |
| NFE2L2 | NM_006164 |
| NFRKB | NM_006165 |
| NGFB | NM_002506 |
| NTF3 | NM_002527 |
| NUMA1 | NM_006185 |
| TNRC11 | NM_005120 |
| SLC22A1L | NM_002555 |
| DUSP2 | NM_004418 |
| PAFAH2 | NM_000437 |
| PAPPA | NM_002581 |
| PCM1 | NM_006197 |
| PCTK1 | NM_006201 |
| PDE4A | NM_006202 |
| PDE4B | NM_002600 |
| PEX10 | NM_002617 |
| SERPINB9 | NM_004155 |
| PIGA | NM_002641 |
| PLAGL1 | NM_002656 |
| POU2AF1 | NM_006235 |
| PRKG1 | NM_006258 |
| MAPK10 | NM_002753 |
| MAPK9 | NM_002752 |
| PROP1 | NM_006261 |
| PSD | NM_002779 |
| PTK2B | NM_004103 |
| PTN | NM_002825 |
| PTPN13 | NM_006264 |
| PTPN6 | NM_002831 |
| PTPRD | NM_002839 |
| PTPRG | NM_002841 |
| QDPR | NM_000320 |
| RAC3 | NM_005052 |
| RELA | NM_021975 |
| REQ | NM_006268 |
| RMSA1 | NM_002932 |
| RSN | NM_002956 |
| S100A7 | NM_002963 |
| S100A8 | NM_002964 |
| IQGAP1 | NM_003870 |
| SCN1B | NM_001037 |
| SCN5A | NM_000335 |
| SCNN1G | NM_001039 |
| SCYA14 | NM_004166 |
| SCYA7 | NM_006273 |
| SDHC | NM_003001 |
| SELPLG | NM_003006 |
| SFTPA2 | NM_006926 |
| SGSH | NM_000199 |
| SHMT2 | NM_005412 |
| MYH11 | NM_002474 |
| SNRPN | NM_003097 |
| SOAT1 | NM_003101 |
| SORL1 | NM_003105 |
| SPP1 | NM_000582 |
| SSTR1 | NM_001049 |
| STATI2 | NM_003877 |
| STX1B | NM_003163 |
| TCF8 | NM_030751 |
| TCP1 | NM_030752 |
| TF | NM_001063 |
| TGFBI | NM_000358 |
| TGFBR3 | NM_003243 |
| TGM2 | NM_004613 |
| TLR1 | NM_003263 |
| TM4SF7 | NM_003271 |
| TNFAIP6 | NM_007115 |
| TNFRSF1A | NM_001065 |
| TNFSF12 | NM_003809 |
| TPH | NM_004179 |
| TPI1 | NM_000365 |
| TRAF2 | NM_021138 |
| TRAF5 | NM_004619 |
| TSTA3 | NM_003313 |
| TTR | NM_000371 |
| UBE1 | NM_003334 |
| UBE2V2 | NM_003350 |
| UMPK | NM_012474 |
| UP | NM_003364 |
| UPK1B | NM_006952 |
| USP7 | NM_003470 |
| VASP | NM_003370 |
| VDR | NM_000376 |
| NSEP1 | NM_004559 |
| ZFP161 | NM_003409 |
| AQP1 | NM_000385 |
| BDKRB1 | NM_000710 |
| F13A1 | NM_000129 |

1. A nucleic acid comprising an at a least 18 bases-long sequence segment of the chemically pretreated DNA of genes associated with the immune system according to one of the Seq. ID No.1 through Seq. ID No.2420 and sequences complementary thereto.

2. A nucleic acid comprising an at a least 18 bases-long sequence segment of the chemically pretreated DNA of genes associated with the immune system according to one of the sequences of the genes A1BG (T80683), C4A (K02403), C4BPAL2 (X81360), CD1A (M27735), CD20 (L23418), CDR2 (M63256), CENPB (X05299), COL11A2 (U32169), CR1L (M31230), CYP2A (X06401), EBF (AA504812), ERCC2 (L47234), ESD (M13450), ETV4 (D12765), FCGR2C (M90737), FLG (M24355), FN1 (M10905), ITGA1 (X68742), ITGAD (U40274), KRT4 (X07695), KSR (U43586), LY9 (L42621), MEKK1 (U29671), MFAP4 (L38486), MMP18 (Y08622), MYCL1 (M19720), NOTCH1 (M73980), PDE7A (L12052), PIK3R1 (M61906), SLC9A1 (M81768), TCF3 (M31222), TCRA (M12959), TCRB (K02779), TCRG (M27331), TLR5 (U08888), TNFSF11 (AF013171), UBC (AB009010), ZNF121 (M99593), ZRK (L08961), ALPPL2 (NM_{—}031313), AHSG (NM_{—}001622), FCGR3A (NM_{—}000569), FUT3 (NM_{—}000149), IL1R2 (NM_{—}004633), IL2RB (NM_{—}000878), LHB (NM_{—}000894), MDH2 (NM_{—}005918), SLC11A2 (NM_{—}000617), OMG (NM_{—}002544), PIK3CA (NM_{—}006218), TPM1 (NM_{—}000366), TUB (NM_{—}003320), ABAT (NM 000663), ACADL (NM_{—}001608), ACO1 (NM_{—}002197), ADAM10 (NM_{—}001110), ADD1 (NM_{—}014189), ADH4 (NM_{—}000670), ADRA2C (NM_{—}000683), AGA (NM_{—}000027), AGTR2 (NM_{—}000686), AKT1 (NM_{—}005163), ALDH6 (NM_{—}000693), AMPH (NM_{—}001635), ANXA4 (NM_{—}001153), APBA2 (NM_{—}005503), APC (NM_{—}000038), APOA2 (NM_{—}001643), ARHGAP1 (NM_{—}004308), ATOX1 (NM_{—}004045), ATP2B2 (NM_{—}001683), ATP4B (NM_{—}000705), ATR (NM_{—}001184), AUH (NM_{—}001698), AXL (NM_{—}001699), BCL2 (NM_{—}000633), BENE (NM_{—}005434), BID (NM_{—}001196), BMI1 (NM_{—}005180), BN51T (NM_{—}001722), BUB1 (NM_{—}004336), C1R (NM_{—}001733), C4BPB (NM_{—}000716), C5R1 (NM_{—}001736), CASP3 (NM_{—}004346), CASP7 (NM_{—}001227), CBFB (NM_{—}001755), CCR4 (NM_{—}005508), CD151 (NM_{—}004357), CD36L1 (NM_{—}005505), CD4 (NM_{—}000616), CD81 (NM_{—}004356), CDH12 (NM_{—}004061), CDW52 (NM_{—}001803), CEL (NM_{—}001807), CES1 (NM_{—}001266), CGA (NM_{—}000735), CHS1 (NM_{—}000081), CLDN3 (NM_{—}001306), CNK (NM_{—}004073), CSF2RA (NM_{—}006140), CTSK (NM_{—}000396), CX3CR1 (NM_{—}001337), CYBB (NM_{—}000397), CYP11A (NM_{—}000781), DCC (NM_{—}005215), DFFB (NM_{—}004402), DOCK1 (NM_{—}001380), DPYD (NM_{—}000110), ELAVL2 (NM_{—}004432), ELAVL4 (NM_{—}021952), EPB41 (NM_{—}004437), EPHA3 (NM_{—}005233), EPHX2 (NM_{—}001979), EPS15 (NM_{—}001981), ETV6 (NM_{—}001987), F2 (NM_{—}000506), F8A (NM_{—}012151), FABP6 (NM_{—}001445), FADD (NM_{—}003824), FANCE (NM_{—}021922), FCAR (NM_{—}002000), FGA (NM_{—}021871), FGB (NM_{—}005141), FGFR3 (NM_{—}000142), FGG (NM_{—}000509), HFL3 (NM_{—}005666), FOXO1A (NM_{—}002015), ADAM2 (NM_{—}001464), FUCA1 (NM_{—}000147), FUT2 (NM_{—}000511), FY (NM_{—}002036), GABRA5 (NM_{—}000810), GABRA6 (NM_{—}000811), GAS (NM_{—}000805), GAS6 (NM_{—}000820), GBA (NM_{—}000157), GFI1 (NM_{—}005263), GH2 (NM_{—}002059), GHR (NM_{—}000163), GIF (NM_{—}005142), GNAQ (NM_{—}002072), GP9 (NM_{—}000174), GPR15 (NM_{—}005290), GPR30 (NM_{—}001505), GRB14 (NM_{—}004490), GRIK1 (NM_{—}000830), GUCY2D (NM_{—}000180), HADHA (NM_{—}000182), NRG1 (NM_{—}013964), HIVEP1 (NM_{—}002114), HLALS (NM_{—}001531), HLCS (NM_{—}000411), HMX1 (NM_{—}018942), HNRPD (NM_{—}002138), HSA277165 (NM_{—}018411), HRG (NM_{—}000412), HSPG2 (NM_{—}005529), HTN3 (NM_{—}000200), HTR2A (NM_{—}000621), HTR7 (NM_{—}000872), IFNA1 (NM_{—}024013), IL10RA (NM_{—}001558), IL1A (NM_{—}000575), IL1B (NM_{—}000576), IL1R1 (NM_{—}000877), IL3RA (NM_{—}002183), IL9 (NM_{—}000590), ILF1 (NM_{—}004514), ILF2 (NM_{—}004515), SCYB10 (NM_{—}001565), INPP5D (NM_{—}005541), ITGAX (NM_{—}000887), ITGB1 (NM_{—}002211), ITGB3 (NM_{—}000212), ITGB5 (NM_{—}002213), ITGB7 (NM_{—}000889), ITK (NM_{—}005546), KCNJ3 (NM_{—}002239), KPNA1 (NM_{—}002264), LECT2 (NM_{—}002302), LEPR (NM_{—}002303), LPA (NM_{—}005577), KCNH2 (NM_{—}000238), LSP1 (NM_{—}002339), LTF (NM_{—}002343), MAB21L1 (NM_{—}005584), MAL (NM_{—}002371), MASP1 (NM_{—}001879), MCF2 (NM_{—}005369), MAP3K3 (NM_{—}002401), MMP16 (NM_{—}022564), MMP17 (NM_{—}016155), MMP23A (NM_{—}004659), MMP7 (NM_{—}002423), MYC (NM_{—}002467), NAGA (NM_{—}000262), NAT2 (NM_{—}000015), NDUFS2 (NM_{—}004550), NEB (NM_{—}004543), NEU1 (NM_{—}000434), NFATC4 (NM_{—}004554), NFE2L2 (NM_{—}006164), NFRKB (NM_{—}006165), NGFB (NM_{—}002506), NTF3 (NM_{—}002527), NUMA1 (NM_{—}006185), TNRC11 (NM_{—}005120), SLC22A1L (NM_{—}002555), DUSP2 (NM_{—}004418), PAFAH2 (NM_{—}000437), PAPPA (NM_{—}002581), PCM1 (NM_{—}006197), PCTK1 (NM_{—}006201), PDE4A (NM_{—}006202), PDE4B (NM_{—}002600), PEX10 (NM_{—}002617), SERPINB9 (NM_{—}004155), PIGA (NM_{—}002641), PLAGL1 (NM_{—}002656), POU2AF1 (NM_{—}006235), PRKG1 (NM_{—}006258), MAPK10 (NM_{—}002753), MAPK9 (NM_{—}002752), PROP1 (NM_{—}006261), PSD (NM_{—}002779), PTK2B (NM_{—}004103), PTN (NM_{—}002825), PTPN13 (NM_{—}006264), PTPN6 (NM_{—}002831), PTPRD (NM_{—}002839), PTPRG (NM_{—}002841), QDPR (NM_{—}000320), RAC3 (NM_{—}005052), RELA (NM_{—}021975), REQ (NM_{—}006268), RMSA1 (NM_{—}002932), RSN (NM_{—}002956), S100A7 (NM_{—}002963), S100A8 (NM_{—}002964), IQGAP1 (NM_{—}003870), SCN1B (NM_{—}001037), SCN5A (NM_{—}000335), SCNN1G (NM_{—}001039), SCYA14 (NM_{—}004166), SCYA7 (NM_{—}006273), SDHC (NM_{—}003001), SELPLG (NM_{—}003006), SFTPA2 (NM_{—}006926), SGSH (NM_{—}000199), SHMT2 (NM_{—}005412), MYH11 (NM_{—}002474), SNRPN (NM_{—}003097), SOAT1 (NM_{—}003101), SORL1 (NM_{—}003105), SPP1 (NM_{—}000582), SSTR1 (NM_{—}001049), STATI2 (NM_{—}003877), STX1B (NM_{—}003163), TCF8 (NM_{—}030751), TCP1 (NM_{—}030752), TF (NM_{—}001063), TGFBI (NM_{—}000358), TGFBR3 (NM_{—}003243), TGM2 (NM_{—}004613), TLR1 (NM_{—}003263), TM4SF7 (NM_{—}003271), TNFAIP6 (NM_{—}007115), TNFRSF1A (NM_{—}001065), TNFSF12 (NM_{—}003809), TPH (NM_{—}004179), TPI1 (NM_{—}000365), TRAF2 (NM_{—}021138), TRAF5 (NM_{—}004619), TSTA3 (NM_{—}003313), TTR (NM_{—}000371), UBE1 (NM_{—}003334), UBE2V2 (NM_{—}003350), UMPK (NM_{—}012474), UP (NM_{—}003364), UPK1B (NM_{—}006952), USP7 (NM_{—}003470), VASP (NM_{—}003370), VDR (NM_{—}000376), NSEP1 (NM_{—}004559), ZFP161 (NM_{—}003409), AQP1 (NM_{—}000385), BDKRB1 (NM_{—}000710), F13A1 (NM_{—}000129), and complementary sequences thereof.

3. An oligomer (oligonucleotide or peptide nucleic acid (PNA)-oligomer) for detecting the cytosine methylation status in chemically pretreated DNA, comprising in each case at least one base sequence having a length of at least 9 nucleotides which hybridises to a chemically pretreated DNA of genes associated with the immune system according to one of the Seq. ID No.1 through Seq. ID No.2420 according to claim 1 or to a chemically pretreated DNA of genes according to claim 2 and to the complementary sequences thereof.

4. The oligomer according to claim 3, wherein the base sequence comprises at least one CpG dinucleotide.

5. The oligomer as recited in claim 3, characterised in that the cytosine of the CpG dinucleotide is located approximately in the middle third of the oligomer.

6. A set of oligomers comprising at least two oligomers according to one of the claims 3 to 5.

7. The set of oligomers according to claim 6 comprising oligomers for the detection of the methylation status of all CpG dinucleotides from one of the sequences Seq. ID 1 through Seq. ID 2420 according to claim 1 or from a chemically pretreated DNA from genes according to claim 2 and complementary sequences thereof.

8. The set of at least two oligonucleotides according to claim 3, for the amplification of DNA sequences of a sequence from one of the Seq. ID 1 through Seq. ID 2420 and complementary sequences therof, and/or sequences of a chemically pretreated DNA from genes according to claim 2 and complementary sequences or segments thereof.

9. A set of oligonucleotides according to claim 8, characterised in that at least one oligonucleotide is bonded to a solid phase.

10. A set of oligomer probes, comprising at least ten oligomers according to one of the claims 6 to 9, for the detection of the cytosine methylation state and/or single nucleotide polymorphisms (SNPs) in chemically pretreated genomic DNA according to claim 1 or a chemically pretreated DNA from genes according to claim 2.

11. A method for manufacturing an arrangement of different oligomers (array) fixed to a carrier material for the analysis of [diseases] associated with the methylation state of the CpG dinucleotides of one of the Seq. ID 1 through Seq. ID 2420 and to sequences complementary thereof [and/or oligonucleotide-] and/or chemically pretreated DNA from genes according to claim 2, werein at least one oligomer according to one of the claims 3 to 5 is coupled to a solid phase.

12. An arrangement of different oligomers (array), bonded to a solid phase, according to claim 11.

13. An array of different oligonucleotide- and/or PNA-oligomer sequences according to claim 12, characterised in that these are arranged on a plane solid phase in the form of a rectangular or hexagonal lattice.

14. The array according to claims 12 or 13, characterised in that the solid phase surface is composed of silicon, glass, polystyrene, aluminium, steel, iron, copper, nickel, silver, or gold.

15. A DNA- and/or PNA-array for analysing diseases associated with the methylation status of genes, comprising at least one nucleic acid according to one of the preceding claims.

16. A method for ascertaining genetic and/or epigenetic parameters for the diagnosis and/or therapy of existing diseases or the predisposition for specific diseases by analysing cytosine methylations, characterised in that the following steps are carried out:
in a genomic DNA sample, cytosine bases which are unmethylated at the 5-position are converted, by chemical treatment, to uracil or another base which is dissimilar to cytosine in terms of hybridisation behaviour;
fragments from this chemically pretreated genomic DNA are amplified using sets of primer oligonucleotides according to claim 8 or 9 and a polymerase, the amplificates carrying a detectable label;
amplificates are hybridised to a set of oligonucleotides and/or PNA probes according to the claims 6 to 7, or else to an array according to one of the claims 12 to 15;
the hybridised amplificates are subsequently detected.

17. The method according to claim 16, characterised in that the chemical treatment is carried out by means of a solution of a bisulfite, hydrogen sulfite or disulfite.

18. The method according to one of the claims 16 or 17, characterised in that more than ten different fragments having a length of 100-2000 base pairs are amplified.

19. The method according to one of the claims 16 to 18, characterised in that the amplification of several DNA segments is carried out in one reaction vessel.

20. The method according to one of the claims 16 to 19, characterised in that the polymerase is a heat-resistant DNA polymerase.

21. The method according to claim 20, characterised in that the amplification is carried out by means of polymerase chain reaction (PCR).

22. The method according to one of the claims 16 to 21, characterised in that the labels of the amplificates are fluorescence labels.

23. The method method according to one of the claims 16 to 21, characterised in that the labels of the amplificates are radionuclides.

24. The method according to one of the claims 16 to 21, characterised in that the labels of the amplificates are detachable molecule fragments having a typical mass which are detected in a mass spectrometer.

25. The method according to one of the claims 16 to 21, characterised in that the amplificates or fragments of the amplificates are detected in the mass spectrometer.

26. The method according to one of the claims 24 and/or 25, characterised in that the produced fragments have a single positive or negative net charge for better detectability in the mass spectrometer.

27. The method according to one of the claims 24 to 26, characterised in that detection is carried out and visualized by means of matrix assisted laser desorption/Ionization mass spectrometry (MALDI) or using electron spray mass spectrometry (ESI).

28. The method according to one of the claims 16 to 27, characterised in that the genomic DNA is obtained form cells or cell components containing DNA, the sources of DNA comprising, for example, cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, and all possible combinations thereof.

29. A kit comprising a bisulfite (=disulfite, hydrogen sulfite) reagent as well as oligonucleotides and/or PNA-oligomers according to one of the claims 3 to 5.

30. The use of a nucleic acid according to one of the claims 1 or 2, of an oligonucleotide or PNA-oligomer according to one of the claims 3 through 5, of a kit according to claim 29, of an array according to one of the claims 12 through 15, of a set of oligonucleotides according to one of the claims 6 to 9, for the diagnosis of diseases associated with the immune system.

31. The use of a nucleic acid according to one of the claims 1 or 2, of an oligonucleotide or PNA-oligomer according to one of the claims 3 to 5, of a kit according to claim 29, of an array according to one of the claims 12 to 15, of a set of oligonucleotides according to one of the claims 6 to 9, for the therapy of diseases associated with the immune system.
